(19) United States
(12) Patent Application Publication
(10) Pub. No.: US 2008/0009693 A1
(43) Pub. Date: Jan. 10, 2008

(54) Moisture control in a transdermal blood alcohol monitor

(76) Inventors: Jeffrey Scott Hawthorne, Bennett, CO (US); Mark Henry Wojcik, Littleton, CO (US); William James Roushey, Littleton, CO (US); David Cain Collins, Denver, CO (US)

Correspondence Address: MERCHANT & GOULD PC, P.O. BOX 2903, MINNEAPOLIS, MN, 55402-0903, US

(21) Appl. No.: 11/454,491

(22) Filed: Jun. 16, 2006

(51) Int. Cl. A61B 5/00

(52) U.S. Cl. 600/364

(57) ABSTRACT

Moisture buildup inside an alcohol monitor that is securely attached to a human subject is due to the inlet air from the subject's skin surface, which constantly emits water vapor in the form of insensible skin perspiration. As the warm moist air flows along the air flow path through decreasing temperatures within the alcohol monitor, moisture will be removed from the air through condensation. The present invention solves this condensation problem by first simplifying the air flow path, eliminating barriers that can trap water. Second, additional changes to the air flow path take advantage of gravity, allowing water to drain out of the alcohol monitor. Third, by better balancing the volume of air sample between the sample collection chamber and the fuel cell sample chamber, the total volume of air taken in is reduced, resulting in an overall reduction in the volume of potential moisture introduced into the alcohol monitor.

### FIELD OF THE INVENTION

This invention relates to transdermal blood alcohol monitors for continuous monitoring of blood alcohol levels, and more particularly, the invention relates to improved moisture control within a transdermal blood alcohol monitor or similar device.

### BACKGROUND OF THE INVENTION

Reference is made to U.S. Pat. No. 5,220,919 titled “BLOOD ALCOHOL MONITOR” European Patent No. EPO 623001B1 titled “BLOOD ALCOHOL MONITOR”, and U.S. patent application Ser. No. 10/441,940 titled “METHOD AND APPARATUS FOR REMOTE BLOOD ALCOHOL MONITORING,” all owned by the assignee of this invention and all are incorporated herein by reference in their entirety for all that is taught and disclosed therein.

Individuals on probation, parole, or in alcohol treatment programs may be prohibited from consuming alcohol, and many federal, state, and local law enforcement agencies require testing to ensure participants in court ordered programs remain alcohol free. In general, present-generation remote alcohol monitoring devices used in probation, parole, and treatment settings are fixed-location breath-testing devices that measure Blood Alcohol Content (“BAC”) and incorporate voice or video identification of the participant. If a subject tests positive for alcohol, the monitoring device then sends a message alerting the monitoring center of a violation by the subject, and the monitoring center then sends an alert message to the subject's supervising agency or dedicated administrator.

As alcohol is ingested orally, it is absorbed into the body's blood and distributed throughout the body via the circulatory system. Alcohol is eliminated from the body by two mechanisms: metabolism and excretion. Metabolism accounts for the removal of greater than 90% of the alcohol consumed, removing it from the body via oxidation of the ethyl alcohol molecule to carbon dioxide and water primarily in the liver. The remaining alcohol is excreted unchanged wherever water is removed from the body—breath, urine, insensible skin perspiration, and saliva. Although excretion accounts for less than 10% of the eliminated alcohol, it is significant because unaltered alcohol excretion permits an accurate measurement of alcohol concentration in the body by way of both breath analysis and insensible skin perspiration. Insensible skin perspiration is the vapor that escapes through the skin through sweating. The average person will emit approximately one liter of insensible skin perspiration each day. This insensible skin perspiration can be used to obtain a transdermal measurement of blood alcohol concentration, referred to as Transdermal Alcohol Concentration (“TAC”).

Transdermal monitoring of blood alcohol levels is accomplished by taking percentage measurements of alcohol contained in the insensible skin perspiration that is expelled transdermally through human skin. Throughout this description of the invention, insensible skin perspiration may be referred to as “vapor,” “air vapor,” “air vapor sample,” “air vapor volume,” “sample,” “sample volume,” “air sample,” and “air sample volume,” interchangeably, with no difference in meaning intended. A monitoring device is attached to the skin to capture the air vapor and measure the alcohol contained therein, if any.

There are numerous advantages to transdermal alcohol monitoring, as opposed to breath-testing, including, but not limited to, the ability to take readings at any time without the knowledge of the subject, consistent and continuous testing (unlike breath alcohol testing where a subject breathing incorrectly into the testing device can cause inaccurate results), and the ability to convert such readings into electrical signals that can be transmitted to a central monitoring station.

However, there is a need to better manage the build-up of moisture within a transdermal blood alcohol monitor to prevent damage to the various internal components, and to increase the service life of the transdermal blood alcohol monitor. The present invention meets these and other needs in the art.

### BRIEF DESCRIPTION OF THE DRAWINGS

FIG. 1 shows a block diagram of an alcohol monitoring system of the present invention.

FIG. 2 shows a perspective view of the alcohol monitor of the present invention.

FIG. 3 shows an exploded perspective view of the analog side of the alcohol monitor shown in FIG. 2.

FIG. 4A shows an elevation view of the analog side of the alcohol monitor shown in FIG. 2 in an embodiment of the present invention.

FIG. 4B shows a cross-sectional view taken about line B-B of FIG. 4A in an embodiment of the present invention.

FIG. 4C shows a cross-sectional view taken about line C-C of FIG. 4A in an embodiment of the present invention.

FIG. 4D shows a detailed view taken at D of FIG. 4B in an embodiment of the present invention.

FIG. 4E shows a top view of the alcohol monitor shown in FIG. 2 in an embodiment of the present invention.

FIG. 5 shows the air flow path and the relationship between the intake volume and sample volume of the alcohol monitor of the present invention.

FIG. 6 shows a graph depicting the effect on TAC readings compared to actual BAC readings when the volume of V_{INLET} is equal to five times that of V_{CELL}.

### DETAILED DESCRIPTION OF THE INVENTION

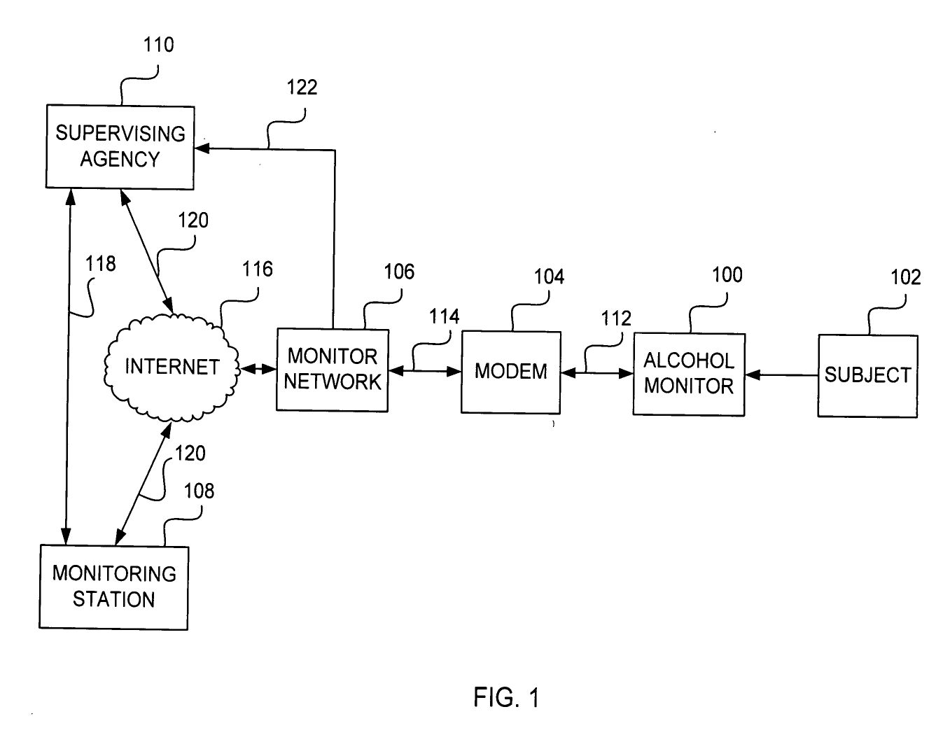

Referring now to the Figures, in which like reference numerals refer to structurally and/or functionally similar elements thereof, FIG. 1 shows by way of illustrative example a system block diagram of remote blood alcohol monitoring between a human Subject 102 and a Monitoring Station 108 utilizing Alcohol Monitor 100 of the present invention. In one embodiment, Alcohol Monitor 100 weighs about eight ounces, is waterproof, designed to handle the stress of everyday activity, and can be worn under any conditions, including bathing and swimming. Alcohol Monitor 100 is attached to the Subject 102. Once Alcohol Monitor 100 is in place, it cannot be removed without triggering a tamper alarm, which is recorded in Alcohol Monitor 100. In addition, there are a number of anti-tamper features designed into Alcohol Monitor 100 to ensure that the TAC readings taken are from Subject 102, and accurately represent the blood alcohol level of Subject 102 and not some other person. Though this discussion focuses on one Subject 102, one skilled in the art will recognize that many Alcohol Monitors 100 may be attached to many Subjects 102 at the same time over a broad geographic area, and all may be monitored by Monitoring Station 108, which is the intended purpose. Likewise, there may be multiple Monitor Networks 106 and Monitoring Stations 108 that manage additional Subjects 102 in diverse geographic locations.

Alcohol Monitor 100 will take TAC readings that are time stamped at predetermined or random intervals twenty-four hours a day, seven days a week, 365 days a year, without active participation by Subject 102. Testing schedules may range from as frequent as every 30 minutes or as infrequent as once per day. Alcohol Monitor 100 collects TAC data from Subject 102 regardless of the location or activity of Subject 102. While commuting, at work, at home, during recreation, in the shower, or sleeping, Subject 102 is passively monitored, allowing for continual, effective monitoring while Subject 102 maintains a normal routine. Subject 102 typically does not know when the sampling will occur. Typical existing alcohol monitoring programs that have used other means of testing subjects for alcohol will likely see an increase in the number of program positives utilizing the present invention. This is a result of the continuous monitoring, rather than the pre-arranged, specific testing times typical of current monitoring programs. Continuous monitoring eliminates the ability for subjects to manipulate their drinking patterns to avoid detection.

TAC readings are taken as scheduled without the participation of Subject 102, with the data uploaded at scheduled time intervals to Modem 104, or immediately if a positive drinking event or a tamper is detected and Modem 104 is in range. Typically, Modem 104 would be placed at the residence of Subject 102, and Subject 102 is merely required to periodically be in proximity to Modem 104 for the purpose of allowing automatic transmission of TAC measurements taken by Alcohol Monitor 100 over a period of time. Subject 102 comes within range of Modem 104, typically within about ten to twenty feet, on a periodic basis, such as once per day, to allow the automatic transmission to take place. Different hardware components may increase or decrease the range at which the automatic transmission will take place. Subject 102 may rise and leave for work, return home, and remain at home until the next day when it is time to leave for work again. When Alcohol Monitor 100 is in range and the timer indicates that it is time to communicate with Modem 104, Alcohol Monitor 100 will transfer to Modem 104 through radio frequency (“RF”) signals through bi-directional RF Communication Link 112 all the TAC readings, tamper indicators, error indicators, diagnostic data, and any other data stored in Alcohol Monitor 100 regarding Subject 102. Modem 104 also can transmit operational information, such as monitoring schedules and reporting schedules in the form of RF signals back to Alcohol Monitor 100 over bi-directional RF Communication Link 112.

Modem 104 stores the data contained in the RF signals received from Alcohol Monitor 100 for transmission to Monitor Network 106. After receiving all of the information from Alcohol Monitor 100, Modem 104 will check the stored data for any TAC readings, tampers, errors, or diagnostic data. Any one of these, or a trigger from a predetermined time interval, will cause Modem 104 to establish a connection over Communication Link 114 with Monitor Network 106. Once a connection is established, Monitor Network 106 validates the identity of Modem 104 and authenticates the data before it is stored. Once validated, Modem 104 will transfer all of the TAC readings, tampers, errors, diagnostic data, and any other data stored to a web-hosted database server at Monitor Network 106 where all data is permanently stored. Monitor Network 106 then analyzes the data received and separates and groups the data into a number of separate categories for reporting to monitoring personnel at Monitoring Station 108. The data can then be accessed by the monitoring personnel through the use of secured dedicated websites through the Internet 116 and Internet Connection 120 to Monitor Network 106. When Monitor Network 106 analyzes the data received, an automatic alert, based upon a rules-based database, may be sent directly from Monitor Network 106 to a call center at Supervising Agency 110 over Communication Link 122, or to an individual previously designated by Supervising Agency 110, when a specific alert, or combination of alerts, are received. The alert may be an e-mail, a fax, or a page to a previously provided number. Communication Link 122 may be a wire or wireless connection.

Monitor Network 106 may be located at Monitoring Station 108, or in a separate location. Monitoring personnel at Monitoring Station 108 have access to all of the data gathered on all of the Subjects 102. Supervising personnel at the call center of Supervising Agency 110, however, only have access to those Subjects 102 that are associated with Supervising Agency 110.

Monitoring Station 108 may automatically or periodically transmit data received from Modem 104 via Monitor Network 106 to one or more persons at Supervising Agency 110 who are assigned to monitor Subject 102, such as a parole officer, probation officer, case worker, or other designated person or persons in charge of enrolling Subject 102 and monitoring the data being collected on Subject 102. Only one Supervising Agency 110 is shown for simplicity, but one skilled in the art will recognize that many Supervising Agencies 110 may be accessing Monitor Network 106 at any given time. A connection is established with Supervising Agency 110 through Communication Link 118. Typically this connection is accomplished via the telephone system through a wire or wireless link, and may connect to a pager or cellular phone of the designated person. Designated personnel at Supervising Agency 110 may also access Monitor Network 106 through the use of secured dedicated websites through the Internet 116 and Internet Connection 120 to Monitor Network 106. Monitor Network 106 web software allows Supervising Agency 110 the ability to track Subject 102 compliance in a manner most feasible to them, and can be defined to fit the needs of both small and large programs. Each Supervising Agency 110 may customize the frequency of monitoring and the method of notification for alerts that they want to receive from Monitor Network 106. Alerts may be categorized by the type and severity of alert, allowing each Supervising Agency 110 to prioritize and better categorize a response (i.e., a low battery warning versus a possible alcohol violation).

Each Supervising Agency 110 has its own separate data storage area on the database server at Monitor Network 106 so that representatives from each Supervising Agency 110 can retrieve the secure data they need when they need it. Existing monitoring agencies that are experienced at managing alcohol offenders may easily take advantage of this approach.

Utilizing Alcohol Monitor 100 with the system described has many advantages and benefits over existing methods and apparatus, including, but not limited to, no collection of body fluids (blood, breath, urine) that require special gathering, handling, or disposal considerations; no waiting for laboratory test results; there is no need for the subject to travel to a test center; continuous 24/7/365 monitoring and data collection from any location; no subject, agency official, or laboratory intervention—only passive participation on the part of the subject; the monitoring device is light weight and can be hidden from normal view; tamper-resistant technology ensures accurate readings representative of the subject being monitored; advanced technology utilizing microprocessors, encrypted data links, and secure data storage and retrieval; the ability for monitored subjects to maintain normal daily routines, including work, counseling, community service, family obligations, and recreation; and easy, web-based, secure access for the monitoring agency to each subject's data.

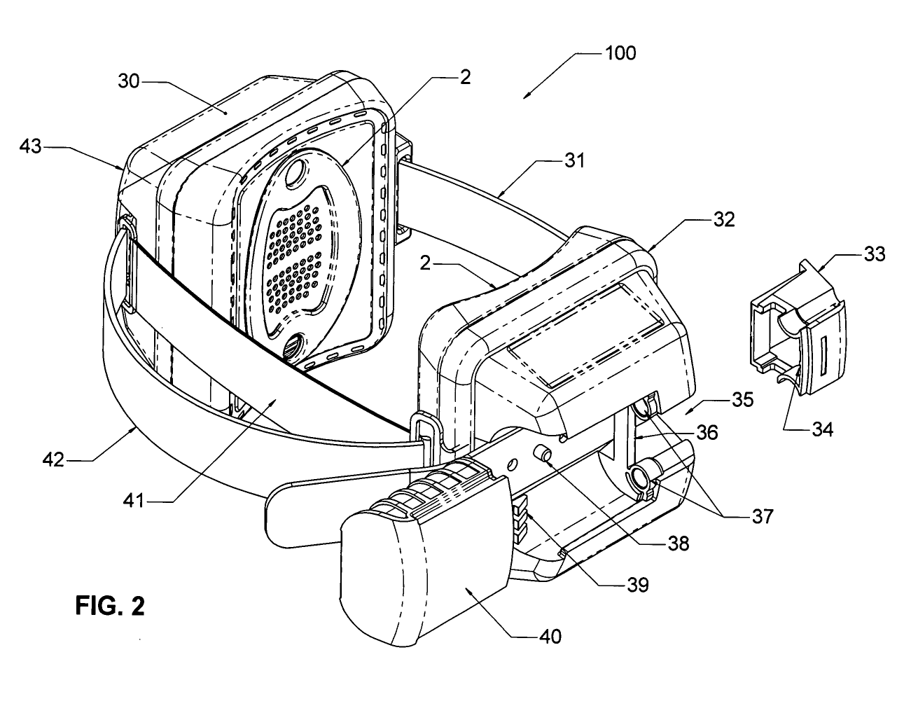

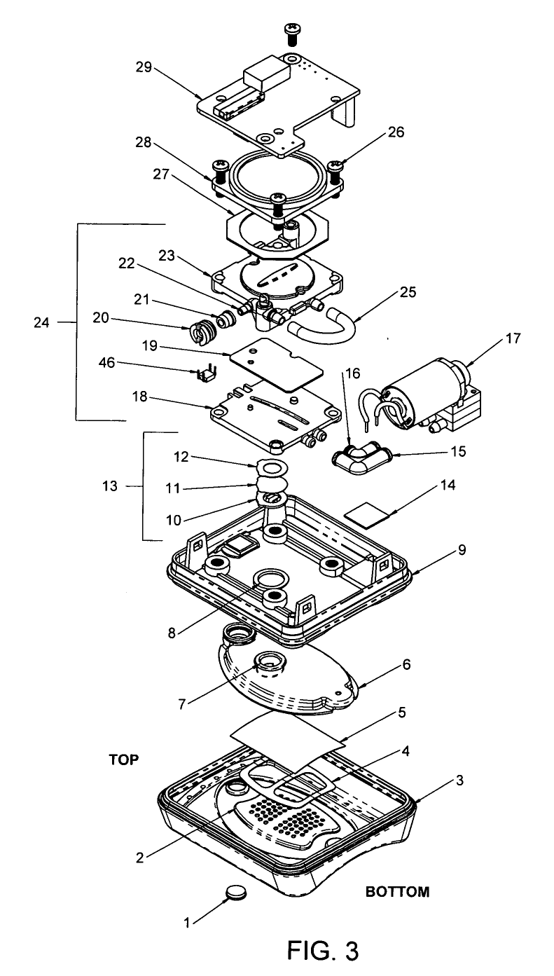

Referring now to FIG. 2, an embodiment of Alcohol Monitor 100 is illustrated for attachment to a human Subject 102. Alcohol Monitor 100 is in the form of a bracelet broadly comprised of an analog side having Analog Housing 43, digital side having Digital Housing 32 and Battery Housing 40, and Elastic Strap 41 with Flexible Circuit 42 connected to Conductive Strap 31 between the Analog Housing 43 and Digital Housing 32, all of which enable the bracelet to encircle the limb of a human Subject 102, such as an arm or a leg. Flexible Circuit 42 contains the circuit connections between an Analog Board 29 (see FIG. 3) in Analog Housing 43 and a digital board in Digital Housing 32. One end of Conductive Strap 31 is connected to Flexible Circuit 42, and the other end of Conductive Strap 31 has a series of holes punched there through which are designed to fit in cooperation with Securing Pins 38 in Enlarged Channel 35 in Digital Housing 32 so that Alcohol Monitor 100 may be adjustably tightened to fit securely to the limb of Subject 102. A Strap Securing Bracket (not visible in FIG. 2) attaches to Analog Housing 43 and channels Conductive Strap 31 towards Digital Housing 32. The rigidity of the Strap Securing Bracket along its length over a portion of Conductive Strap 31 prevents Subject 102 from being able to manipulate and rotate Analog Housing 43 and Digital Housing 32 inside out so that Cover Plates 2 are facing outward from the skin of Subject 102.

Analog Housing 43 is preferably a rigid casing generally rectangular in cross-section with a concave-shaped open interior with Side Walls 30 and a Back Wall having a Channel (not visible in FIG. 2) for mounting Elastic Strap 41, Conductive Strap 31, and Flexible Circuit 42. A Cover Plate (not visible in FIG. 2) is attached to the Back Wall so as to hold the straps permanently in place. Digital Housing 32 includes Enlarged Channel 35 for insertion of Battery Housing 40 through one end of Enlarged Channel 35 into secure engagement with Battery Clip 33, which is mounted in the opposite end of Enlarged Channel 35 between a pair of Battery Contact Sockets 37. A Cross-Member 36 is permanently mounted in Enlarged Channel 35 between Battery Contact Sockets 37 to support Digital Housing 32. Battery Housing 40, contains a Battery (not visible in FIG. 2). Battery Housing 40 and Battery Clip 33 are hollow and of generally rectangular configuration and correspondingly sized so that projecting catches on the ends of Tangs 39 will move into engagement with a molded breakaway on an off-set portion of Lip 34, on an outer end wall of Battery Clip 33 when Battery Housing 40 and Battery Clip 33 are inserted into opposite ends of Enlarged Channel 35. Battery Housing 40 is designed to be permanently affixed in the Back Wall of Digital Housing 32. When thus fixed in place, it is impossible to remove Alcohol Monitor 100 from the limb of Subject 102 without cutting Flexible Circuit 42 or Conductive Strap 31, or otherwise breaking Analog Housing 43, Digital Housing 32, or Battery Clip 33. Nevertheless, when it does become necessary to replace the Battery, or simply to remove Alcohol Monitor 100 from Subject 102, Battery Housing 40 must be removed.

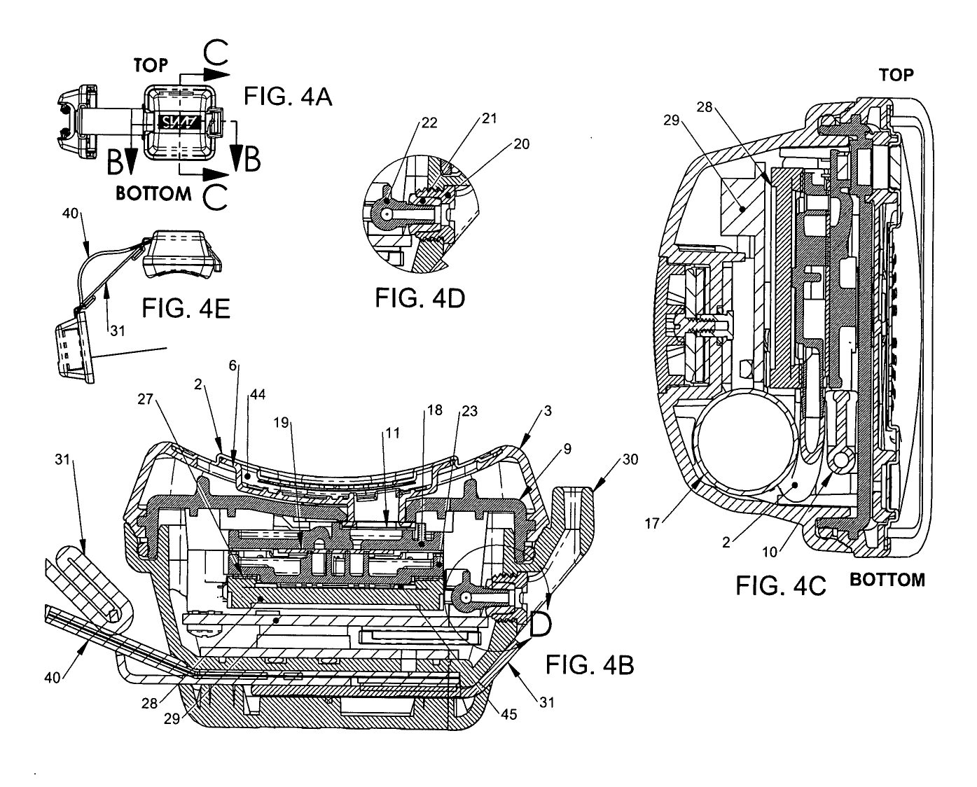

Referring now to FIGS. 3, 4A, 4B, 4C, and 4D, Sample Collection Chamber 44 is formed by Cover Plate 2 and Funnel Plate 6. Hydrophobic Filter 11 is located directly in the path of the air sample flow from Sample Collection Chamber 44 to Pump 17. Pump 17 causes the air sample to flow from Sample Collection Chamber 44 through Lower Manifold 18 and Upper Manifold 23 into Fuel Cell Sample Chamber 45. The air sample will then flow back into Upper Manifold 23 and through Connecting Tube 25, through Tubing Nipple 22 and into Exhaust Port 20 where the air sample is vented to the atmosphere.

Hydrophobic Boot Filter 5 is mounted on Cover Plate 2 located in the bottom of Rubber Boot 3. Cover Plate 2 is curved to conform to the curvature of the leg or arm of human Subject 102 to which it is attached, and is perforated to serve as the inlet for air vapor from the skin of Subject 102 into the interior of Analog Housing 43. Cover Plate 2 is made from surgical stainless steel so as not to cause skin irritation to Subject 102 during the duration of time of continuous wear. Hydrophobic Boot Filter 5 prevents water from directly entering through Cover Plate 2 into Alcohol Monitor 100 so that the subject wearing Alcohol Monitor 100 can bathe, swim, exercise, and engage in other normal life activities without affecting the functioning of Alcohol Monitor 100.

An adhesive Sealing Gasket 4 is used to adhere Hydrophobic Boot Filter 5 to Cover Plate 2. Funnel Plate 6 is mounted to Cover Plate 2 in Rubber Boot 3, enclosing the Hydrophobic Boot Filter 5, and creating Sample Collection Chamber 44. Infrared Lens Filter 1 fits into Cover Plate 2, sealing and water-proofing the infrared sensor path for Infrared Sensor 46. Rubber Boot 3 fits over Base Plate 9, and Nipple 7 on Funnel Plate 6 fits through Hole 8 in Base Plate 9. This arrangement allows the air sample to flow from Sample Collection Chamber 44 into Manifold Assembly 24, which has Lower Manifold 18 and Upper Manifold 23 connected by Adhesive Gasket 19. Sealing Filter Stack 13, composed of Hydrophobic Filter 11 sandwiched between two self adhesive Sealing Gaskets 10 and 12, attaches Lower Manifold 18 to Base Plate 9. Inlet Elbow Tube 15 connects Lower Manifold 18 to Pump 17, which has an internal check valve. A piece of Adhesive Tape 14 is used to secure Pump 17 to Base Plate 9.

Thus, the air sample will flow directly from Sample Collection Chamber 44 through Hydrophobic Filter 11, through Lower Manifold 18, through Inlet Elbow Tube 15 and into Pump 17. The air sample will then pass through Pump 17 into Outlet Elbow Tube 16, back into Lower Manifold 18, through Upper Manifold 23, and into Fuel Cell Sample Chamber 45, which is formed between Fuel Cell 28 and Upper Manifold 23. Sealing Gasket 27 fits between Upper Manifold 23 and Fuel Cell 28.

The air sample flows across the face of Fuel Cell 28 and exits Fuel Cell Sample Chamber 45 through Upper Manifold 23 into Connecting Tube 25, which connects Upper manifold 23 to Tubing Nipple 22. Upon passing through Tubing Nipple 22, the air sample is then exhausted out of Exhaust Port 20 into the atmosphere exterior to Alcohol Monitor 100. A Rubber Grommet 21 is used to attach and seal the connection between the Exhaust Port 20 and the Tubing Nipple 22. Screws 26 secure Analog Board 29 and the entire sample system to Base Plate 9.

In order for Alcohol Monitor 100 to reliably measure blood alcohol content, the insensible skin perspiration which is emitted from the body in the form of air vapor will migrate away from the skin and through Cover Plate 2 and through Hydrophobic Boot Filter 5 of the analog side of Alcohol Monitor 100. These air vapors collect in Sample Collection Chamber 44 between Cover Plate 2 and Funnel Plate 6. Pump 17 is activated to draw the air sample from Sample Collection Chamber 44, through Moisture Filter 11 into Lower Manifold 18, and into Pump 17. The air sample is then forced out of Pump 17 into Lower Manifold 18, where it passes into Upper Manifold 23 and into Fuel Cell Sample Chamber 45, displacing any existing volume of air sample from Fuel Cell Sample Chamber 45. The air sample passes across Fuel Cell 28 generating the TAC signal, and out through Upper Manifold 23 through Connecting Tube 25 and into Tubing Nipple 22. After exiting through Tubing Nipple 22, the air sample will escape into the atmosphere through Exhaust Port 20.

In order to avoid false readings, it is important that Alcohol Monitor 100 be waterproof to prevent the entry of water directly into the air flow path. It is also important that any moisture in the air sample itself be removed, and any water condensation resulting from temperature changes between the point where the air sample enters into Alcohol Monitor 100 to the point where sensor measuring takes place is eliminated or minimized.

A problem encountered with transdermal blood alcohol monitors, such as the transdermal blood alcohol monitor described in U.S. patent application Ser. No. 10/441,940, (hereinafter referred to as the “'940 alcohol monitor”), is moisture build up along the air flow path beginning from the inlet into the alcohol monitor next to the skin of the subject, through the interior of the alcohol monitor, and exiting through the exhaust port. Moisture buildup inside an alcohol monitor is understandable, given that the source of the inlet air is directly from the subject's skin surface, which constantly emits water vapor in the form of insensible skin perspiration. The rate at which moisture builds up inside an alcohol monitor depends in part upon the subject, as each person has a varying amount of perspiration that their body gives off. Condensation of moisture into water droplets within an alcohol monitor can eventually damage internal components, thus reducing the service life of the alcohol monitor. When water buildup is too great within an alcohol monitor, the water may prevent alcohol readings from being taken. This is because alcohol is water soluble, and the fuel cell sensor will not sense the alcohol suspended in water. Alcohol Monitor 100 of the present invention solves these water condensation problems of alcohol monitors.

Laboratory studies have shown that the sample inlet chamber of the '940 alcohol monitor reaches a relative humidity level as high as 95% within the first twenty-four hours of wear by the subject. This high humidity level, along with normal variations in ambient air temperature, creates an environment inside the air flow path that promotes water condensation. In a closed system with 95% humidity, the dew point is within a couple of degrees of the air temperature within the closed system. Dew point temperature is defined as the temperature to which the air would have to cool (at constant pressure and constant water vapor content) in order to reach saturation. A state of saturation exists when the air is holding the maximum amount of water vapor possible at the existing temperature and pressure. When the dew point temperature and air temperature are equal, the air is said to be saturated. If the relative humidity is 100%, the dew point will be equal to the current temperature. As relative humidity falls, the dew point becomes lower, given the same air temperature. Dew point temperature is never greater than the air temperature.

Therefore, if the humidity level in the '940 alcohol monitor is at or near 95%, and there is a temperature difference from four or five or more degrees C. from the air inlet at the boot next to the skin to the fuel cell sample chamber, moisture will be removed from the air through condensation along the air flow path as the warm moist air flows through decreasing temperatures. With each degree drop in temperature, there will be more and more condensation along the air flow path, forming tiny droplets of water within the '940 alcohol monitor.

Alcohol Monitor 100 of the present invention solves this water condensation problem by first simplifying the air flow path by eliminating many of the physical barriers that trap and retain moisture. Second, additional changes made to the air flow path take advantage of gravity, allowing any water droplets that form to flow out of Alcohol Monitor 100 while the subject is in an upright position. Third, by better balancing the volume of air sample between Sample Collection Chamber 44 and Fuel Cell Sample Chamber 45, and reducing the total volume of air taken into Alcohol Monitor 100, an overall reduction in the volume of potential moisture is achieved.

Many of the separate chambers found in the '940 alcohol monitor have been eliminated from Alcohol Monitor 100 in order to achieve a simpler air flow path. Once water droplets formed in the '940 alcohol monitor, they became trapped between the multiple membranes contained therein. Water at times was pulled into the pump, forced out, and sprayed into the fuel cell chamber. The membrane on the exit port also trapped moisture. All of this trapped moisture over time tended to damage various internal components. Trapped moisture caused corrosion on electrical components and on the pump. Corrosion buildup on the pump eventually caused it to fail. Moisture also caused corrosion on the contacts for the flex circuit connector, eventually causing an electrical failure. Instead of having multiple membranes, forming essentially multiple chambers, Alcohol Monitor 100 of the present invention only has a single membrane located internally along the air flow path, Hydrophobic Filter 11. The only other internal barrier in the air flow path is the built in mechanical check valve within Pump 17.

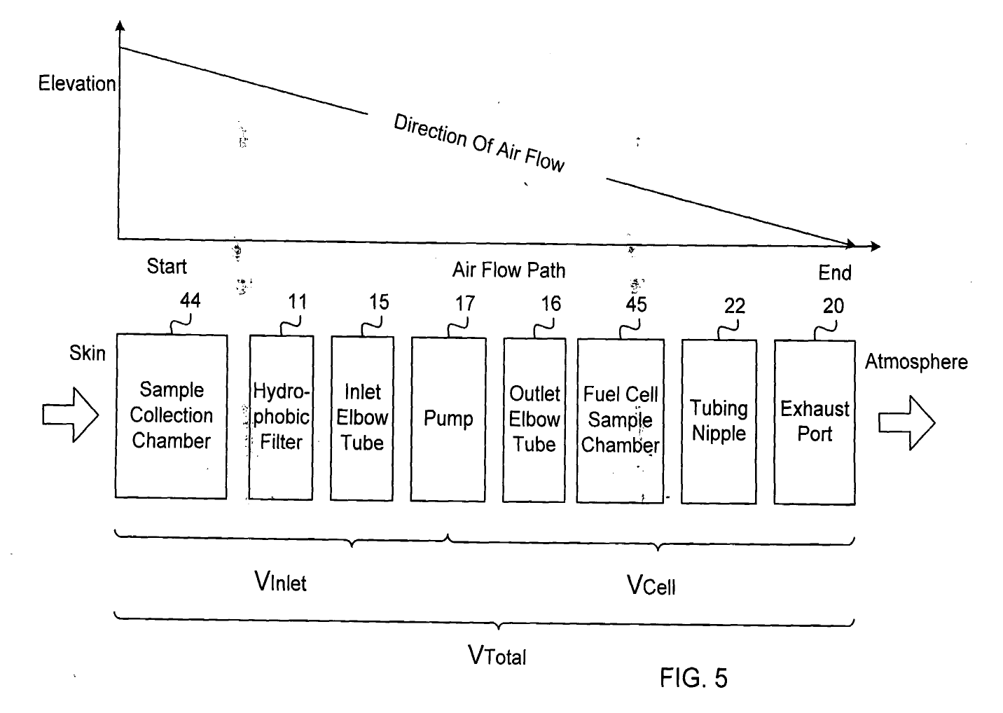

Referring now to FIG. 5, the air flow path of the present invention has been designed so that gravity helps to drain out of Alcohol Monitor 100 any moisture that forms therein. As Alcohol Monitor 100 is situated on the limb of the subject, air vapor is brought into Alcohol Monitor 100, into Sample Collection Chamber 44, and through Funnel Plate 6 in Rubber Boot 3 from a position located on the top side of Rubber Boot 3 (see FIG. 3). As the air passes through Alcohol Monitor 100, it works its way towards the bottom side of Alcohol Monitor 100 through Funnel Plate 6, past Hydrophobic Filter 11, through Inlet Elbow Tube 15, through Pump 17, through Outlet Elbow Tube 16, into Fuel Cell Sample Chamber 45 and across Fuel Cell 28, through Tubing Nipple 22, and then out of Exhaust Port 20 into the ambient air. Any water droplets that do form within Alcohol Monitor 100 will, by gravity, be drawn downward, from the top side of Alcohol Monitor 100 to the bottom side, and drained out of Exhaust Port 20. Therefore, Alcohol Monitor 100 of the current invention must now be oriented on the limb of the subject with a top side oriented up and a bottom side oriented down when the subject is in a standing position. Water may collect in Alcohol Monitor 100 when the subject is lying down, but upon standing, any water droplets formed will begin to drain down and out of Alcohol Monitor 100 due to the force of gravity acting upon the orientation of the air flow path.

Further reduction of the water condensation problem is achieved by reducing the total volume of air sample taken into Alcohol Monitor 100, and by better balancing the volume of air sample between Sample Collection Chamber 44 and Fuel Cell Sample Chamber 45. The total system air volume “V_{TOTAL}” in Alcohol Monitor 100 is first determined by the volume capacity of Fuel Cell Sample Chamber 45, “V_{CELL}”, which is the chamber of Alcohol Monitor 100 where Fuel Cell 28 is exposed to the incoming air sample. The volume of V_{CELL} is the starting point for calculating the air volume for the remainder of the system. The volume of V_{CELL} can include any additional volume used from the internal check valve of Pump 17, through Outlet Elbow Tube 16, through Fuel Cell 28, through Tubing Nipple 22, and through Exhaust Port 20 to the ambient air.

There are two factors for obtaining consistent air sample measurements. First, the volume of Sample Collection Chamber 44, “V_{INLET}”, should be greater than the volume of V_{CELL}. Second, each cycle of the pump should not exceed the volume of V_{CELL}. The V_{INLET} volume can include any additional volume used to route the air sample from the skin, through Sample Collection Chamber 44, through Funnel Plate 6, through Inlet Elbow Tube 15 to the built in mechanical check valve within Pump 17. The check valve within the Pump 17 is necessary in order to separate each new air sample from the previous air sample.

A balanced system is defined as follows:

V_{TOTAL}=V_{INLET}+V_{CELL} where V_{CELL}<V_{INLET}

Optimal readings occur when the ratio of V_{INLET} to V_{CELL} is between:

2V_{CELL}=V_{INLET} up to 4V_{CELL}=V_{INLET}

When V_{INLET} is less than 2V_{CELL}, erratic readings can occur, and the interval between taking fuel cell readings has to be extended. This is because each reading exhausts too much of the air sample. The insensible perspiration being emitted from the skin is not being emitted at a rate fast enough to fill the volume in V_{INLET} before the next reading cycle. Thus, the sample will be depleted and the reading result will be an incorrectly low TAC level. When TAC levels drop below a minimum threshold the interval between readings is extended automatically by the CPU, thus allowing the insensible perspiration to fill the volume in V_{INLET} before the next reading cycle. The next reading will then reflect the correct TAC value.

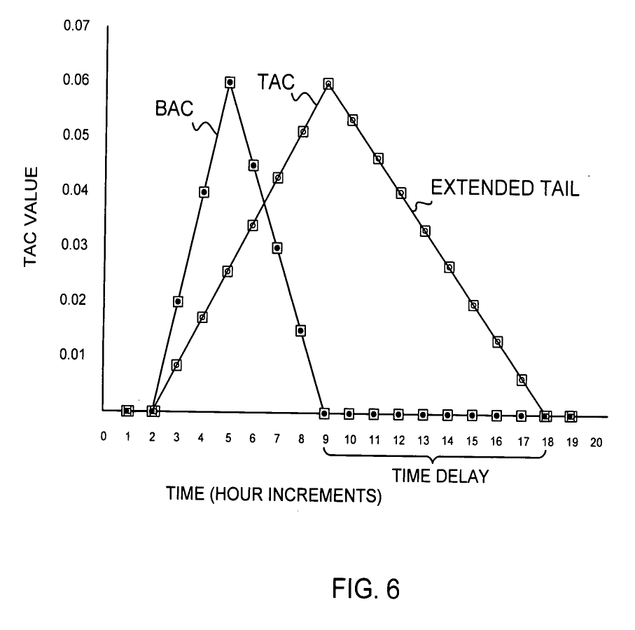

Laboratory analysis has shown that when V_{INLET} is greater than 4V_{CELL}, residual air sample left in the air flow path produces positive readings beyond the actual drinking event duration (an extended “tail” for the drinking event curve). There is a normal lag between a BAC curve and a TAC curve due to the longer amount of time it takes for alcohol to exit the body through insensible skin perspiration. Referring now to FIG. 6, a graph is shown of a normal BAC curve along with the corresponding TAC curve with an extended tail due to the residual air sample left in the air flow path when the V_{INLET} volume of Sample Collection Chamber 44 is five times greater than the V_{CELL} volume of Fuel Cell Sample Chamber 45. In FIG. 6 the volume for V_{INLET} was ten milliliters and the volume for V_{CELL} was two milliliters. This means that Pump 17 had to run a minimum of five cycles to move the entire volume contained in Sample Collection Chamber 44 through Fuel Cell Sample Chamber 45. The effect of this excess sample on the TAC readings is shown in FIG. 6. The normal BAC curve shown in FIG. 6 for a drinking event started and built to a peak in three hours, and returned to zero four hours after the peak. However, the TAC readings from Alcohol Monitor 100 had a delay of nine hours from the time the BAC reached zero until the TAC returned to zero. The TAC curve was extended due to the residual air sample left in the air flow path, extending the readings beyond the actual drinking event. By keeping the ratio of the volume contained in Sample Collection Chamber 44 to that contained in Fuel Cell Sample Chamber 45 to the ratios stated above, the artificially extended tail is eliminated.

The run time for Pump 17 is reduced to match the movement of the volume of air sample equal to V_{CELL}. In other words, for a given flow rate of Pump 17, each pumping cycle is calculated to fully replace a volume of air sample equal to V_{CELL}. The '940 alcohol monitor utilized a ten-to-one fuel cell air sample to inlet air sample ratio, meaning that ten V_{CELL} samples were required to completely evacuate the air sample stored in V_{INLET}. With Alcohol Monitor 100 of the present invention, the sample ratio has been reduced to between two-to-one up to four-to-one. Thus, less air sample is moving through Alcohol Monitor 100, and only two-to-four times the volume of air sample has to be stored in V_{INLET} in order to take TAC readings. The run time for Pump 17 taking a sensor reading has been reduced from approximately 1.4 seconds for the '940 alcohol monitor down to approximately 0.4 seconds for Alcohol Monitor 100 of the present invention.

The face of Fuel Cell 28 in Alcohol Monitor 100 has been opened to the maximum diameter possible to allow the greatest exposure of the sensor surface of Fuel Cell 28 to the incoming air sample. The hydrophobic membrane attached to the check valve assembly in the '940 alcohol monitor that tended to trap condensed water against the alcohol sensor's porous membrane has been eliminated from Alcohol Monitor 100.

Though the invention has been described in terms of its application to a continuous blood alcohol monitoring device, such as Alcohol Monitor 100, one skilled in the art will recognize that the scope of the invention is not so limited. The present invention is applicable to any device that is attached to the body for the purpose of capturing insensible perspiration for analysis. The air coming out of the body is warm and moist and susceptible to water condensation once inside the analysis device. The flow path through the analysis device should be as open as possible with as limited a number of chambers possible, which may tend to collect and entrap condensed water. Optimizing sample volumes and balancing sample volumes helps to prevent collection of water and to prevent water condensation. If water droplets do form, the droplets need to be processed through the system before they get a chance to create a pool of water which may interfere with sensor readings or damage device components. Designing the air path flow so that gravity will assist in draining any water droplets formed out of the analysis device will help mitigate the potential damage caused by water to the internal workings of the device and interference with sensor readings.

Having described the present invention, it will be understood by those skilled in the art that many changes in construction and circuitry and widely differing embodiments and applications of the invention will suggest themselves without departing from the scope of the present invention.

1. (canceled)

2. A method for controlling moisture within a device having an air flow path there through, the method comprising the steps of:
(a) drawing an air vapor sample into the device with a pump through an intake opening having a first hydrophobic filter, wherein said first hydrophobic filter prevents water from entering the device;
(b) drawing said air vapor sample into a sample collection chamber along the air flow path;
(c) drawing said air vapor sample through a second hydrophobic filter along the air flow path; and
(d) drawing said air vapor sample into a fuel cell sample chamber along the air flow path, wherein said sample collection chamber and said fuel cell sample chamber are the only chambers along the air flow path;
wherein said second hydrophobic filter is the only non-movable physical barrier from said first hydrophobic filter located along the air flow path through the device, and further wherein any moisture formed by condensation past said second hydrophobic filter along the air flow path is not physically trapped within the device.

3. The method according to claim 2 further comprising the step of:
placing the device on a limb of a subject prior to said drawing steps.

4. The method according to claim 2 further comprising the step of:
exhausting said air vapor sample out of the device.

5. The method according to claim 2 wherein the device is a transdermal blood alcohol monitor.

6. The method according to claim 5 further comprising the steps of:
performing a reading on said air vapor sample drawn into said fuel cell sample chamber, said performing step further comprising the steps of:
drawing said air vapor sample across a face of a fuel cell within said fuel cell sample chamber; and
generating by said fuel cell a transdermal alcohol concentration signal.

7. The method according to claim 6 further comprising the step of:
repeating said drawing steps and said performing step at predetermined or random time intervals.

8. A device, having moisture control features, that performs readings on air samples, the device comprising:
a body;
an intake opening into said body;
a sample collection chamber within said body;
a first hydrophobic filter between said intake opening and said sample collection chamber;
a fuel cell sample chamber within said body, wherein said sample collection chamber and said fuel cell sample chamber are the only chambers along the air flow path;
an exhaust port out of said body;
an air flow path connecting said intake opening to said sample collection chamber, to said sample chamber, and to said exhaust port;
a second hydrophobic filter between said sample collection chamber and said sample chamber; and
a pump in communication with said air flow path for drawing the air samples into the device through said opening, through said first hydrophobic filter, through said sample collection chamber, through said second hydrophobic filter, through said fuel cell sample chamber, and out of said exhaust port;
wherein said second hydrophobic filter is the only non-movable physical barrier from said first hydrophobic filter located along said air flow path through the device, and further wherein any moisture formed by condensation past said second hydrophobic filter within said air flow path is not physically trapped within the device.

9. The device according to claim 8 wherein the device is a transdermal blood alcohol monitor.

10. The device according to claim 9 further comprising:
a sensor in communication with said fuel cell sample chamber for performing the readings on the air samples, said sensor further comprising:
a fuel cell having a face, wherein the air samples are drawn across said face of said fuel cell, and said fuel cell generates transdermal alcohol concentration signals.

11. The device according to claim 8 wherein said pump is located between said sample collection chamber and said fuel cell sample chamber.

12. The device according to claim 8 further comprising:
an attachment means for attaching the device to a limb of a subject.

13. A method for controlling moisture within a device having an air flow path there through, the method comprising the steps of:
(a) orienting the device so that a bottom side faces down toward the ground, and a top side faces up and away from the ground;
(b) drawing an air vapor sample into the device with a pump through an opening located towards said top side of the device;
(c) pumping said air vapor sample from said opening in a generally downward direction through the air flow path in the device; and
(d) exhausting said air vapor sample with said pump out of an exhaust port located towards said bottom side of the device;
wherein any moisture formed by condensation within the air flow path is drawn by gravity downward through the air flow path and out of said exhaust port.

14. The method according to claim 13 wherein said orienting step further comprises the step of:
placing the device on a limb of a subject so that when said subject is standing, said bottom side of the device faces down toward the ground, and said top side of the device faces up and away from the ground.

15. The method according to claim 13 wherein the device is a transdermal blood alcohol monitor.

16. The method according to claim 15 further comprising the steps of:
performing a reading on said air vapor sample drawn into the device, said performing step further comprising the steps of:
moving said air vapor sample across a face of a fuel cell within the device; and
generating by said fuel cell a transdermal alcohol concentration signal.

17. The method according to claim 16 further comprising the step of:
repeating said moving and generating steps at predetermined or random time intervals.

18. A device, having moisture control features, that performs readings on air samples, the device comprising:
a body having a bottom side that faces down toward the ground and a top side that faces up and away from the ground;
an intake opening located towards said top side of said body;
a sample chamber within said body;
an exhaust port located towards said bottom side of said body;
an air flow path connecting said intake opening to said sample chamber and to said exhaust port; and
a pump in communication with said air flow path for drawing the air sample into the device through said intake opening, through said sample chamber, and out of said exhaust port, all in a generally downward direction from said intake opening to said exhaust port;
wherein any moisture formed by condensation within said air flow path is drawn by gravity downward through said air flow path and out of said exhaust port.

19. The device according to claim 18 further comprising:
an attachment means for attaching the device to a limb of a subject.

20. The device according to claim 18 wherein the device is a transdermal blood alcohol monitor.

21. The device according to claim 20 further comprising:
a sensor in communication with said sample chamber for performing the readings on the air samples, said sensor further comprising:
a fuel cell having a face, wherein the air samples are drawn across said face of said fuel cell, and said fuel cell generates transdermal alcohol concentration signals.

22. The device according to claim 18 further comprising:
a sample collection chamber within said body connected to said intake opening.

23. A method for controlling moisture within a device having an air flow path there through, wherein the air flow path is divided into an inlet volume of air vapor and a sample volume of air vapor, the method comprising the steps of:
(a) balancing a total volume of air vapor such that said total volume of air vapor is equal to a sum of the inlet volume and the sample volume, and further wherein the sample volume is less than the inlet volume; and
(b) reducing an amount of the sample volume required for performing a reading such that between two times the sample volume up to four times the sample volume is equal to the inlet volume;
wherein the balanced said total volume of air vapor in conjunction with the reduced said sample volume minimize an amount of any moisture formed by condensation within the air flow path.

24. The method according to claim 23 further comprising the step of:
placing the device on a limb of a subject before said performing of said reading.

25. The method according to claim 24 wherein the device is a transdermal blood alcohol monitor.

26. The method according to claim 23 further comprising the steps of:
moving the sample volume with a pump across a face of a fuel cell within the device; and
generating by said fuel cell a transdermal alcohol concentration signal.

27. The method according to claim 26 further comprising the step of:
repeating said moving and said generating steps at predetermined or random time intervals.

28. The method according to claim 26 further comprising the step of:
exhausting said air vapor sample with said pump out of the device.

29. A device, having moisture control features, for performing readings on air vapor samples, the device comprising:
a body;
an intake opening into said body;
an exhaust port out of said body;
a pump within said body;
an inlet volume of said body, said inlet volume comprising:
an inlet chamber connected to said intake opening; and
a first air flow path from said inlet chamber to said pump; and
a cell volume of said body, said cell volume comprising:
a fuel cell sample chamber within said body;
a second air flow path from said pump to said fuel cell sample chamber; and
a third air flow path from said fuel cell sample chamber connected to said exhaust port;
wherein in order to balance and reduce a total volume, said inlet volume and said cell volume are sized so that said total volume is equal to a sum of said inlet volume and said cell volume wherein said cell volume is less than said inlet volume, and further wherein between two times said cell volume up to four times said cell volume is equal to said inlet volume, wherein an amount of any moisture formed by condensation within said inlet volume and said cell volume is minimized.

30. The device according to claim 29 further comprising:
an attachment means for attaching the device to a limb of a subject.

31. The device according to claim 30 wherein the device is a transdermal blood alcohol monitor.

32. The device according to claim 31 further comprising:
a sensor in communication with said fuel cell sample chamber for performing the readings on the air vapor samples, said sensor further comprising:
a fuel cell having a face, wherein the air vapor samples are drawn across said face of said fuel cell, and said fuel cell generates transdermal alcohol concentration signals.

33. A method for controlling moisture within a device having an air flow path there through, the method comprising the steps of:
(a) drawing an air vapor sample into the device through a first hydrophobic filter and into a sample collection chamber along the air flow path, wherein said first hydrophobic filter prevents water from entering the device;
(b) drawing said air vapor sample through a second hydrophobic filter and into a fuel cell sample chamber along the air flow path, wherein said second hydrophobic filter is the only non-movable physical barrier from said first hydrophobic filter located along the air flow path through the device, and further wherein any moisture formed by condensation past said second hydrophobic filter along the air flow path is not physically trapped within the device, and further wherein said sample collection chamber and said fuel cell sample chamber are the only chambers along the air flow path;
(c) orienting the device so that a bottom side faces down toward the ground, and a top side faces up and away from the ground, and said air vapor sample is drawn into the device through an opening located towards said top side of the device and exhausted from the device out of an exhaust port located towards said bottom side of the device, wherein said any moisture formed by condensation within the air flow path is drawn by gravity downward through the air flow path and out of said exhaust port;
(d) balancing a total volume of air vapor in the device such that said total volume of air vapor is equal to a sum of an inlet volume and a sample volume, wherein said sample volume is less than said inlet volume; and
(e) reducing an amount of said sample volume required for performing a reading on said sample volume such that between two times said sample volume up to four times said sample volume is equal to said inlet volume, wherein the balanced said total volume of air vapor in conjunction with the reduced said sample volume minimize an amount of said any moisture formed by condensation within the air flow path.

34. The method according to claim 33 wherein the device is a transdermal blood alcohol monitor.

35. The method according to claim 34 further comprising the steps of:
moving said air vapor sample across a face of a fuel cell within said fuel cell sample chamber; and
generating by said fuel cell a transdermal alcohol concentration signal.

36. The method according to claim 33 further comprising the step of:
placing the device on a limb of a subject so that when said subject is standing, said bottom side of the device faces down toward the ground, and said top side of the device faces up and away from the ground.

37. A device, having moisture control features, for performing readings on air vapor samples, the device comprising:
a body having a bottom side that faces down toward the ground and a top side that faces up and away from the ground;
an intake opening located towards said top side of said body;
a sample collection chamber connected to said intake opening;
a first hydrophobic filter between said intake opening and said sample collection chamber;
an exhaust port located towards said bottom side of said body;
a fuel cell sample chamber connecting to said exhaust port;
an air flow path through said body connecting said intake opening, said sample collection chamber, said fuel cell sample chamber, and said exhaust port, wherein said sample collection chamber and said fuel cell sample chamber are the only chambers along the air flow path;
a second hydrophobic filter between said sample collection chamber and said sample chamber, wherein said second hydrophobic filter is the only non-movable physical barrier from said first hydrophobic filter located along said air flow path through the device, and further wherein any moisture formed by condensation past said second hydrophobic filter within the air flow path is not physically trapped within the device;
a pump in communication with said air flow path for drawing the air sample into the device through said intake opening, through said first hydrophobic filter, through said sample collection chamber, through said second hydrophobic filter, through said fuel cell sample chamber, and out of said exhaust port, all in a generally downward direction from said intake opening to said exhaust port, wherein said any moisture formed by condensation within said air flow path is drawn by gravity downward through said air flow path and out of said exhaust port;
an inlet volume of said air flow path, said inlet volume comprising said intake opening and said inlet chamber up to said pump; and
a cell volume of said air flow path, said cell volume comprising said exhaust port and said sample chamber up to said pump;
wherein in order to balance and reduce a total volume of said air flow path, said inlet volume and said cell volume are sized so that said total volume is equal to a sum of said inlet volume and said cell volume, wherein said cell volume is less than said inlet volume, and further wherein between two times said cell volume up to four times said cell volume is equal to said inlet volume, wherein said any moisture formed by condensation within said inlet volume and said cell volume is minimized.

38. The device according to claim 37 wherein the device is a transdermal blood alcohol monitor.

39. The device according to claim 37 further comprising:
a sensor in communication with said fuel cell sample chamber for performing the readings on the air samples, said sensor further comprising:
a fuel cell having a face, wherein the air samples are drawn across said face of said fuel cell, and said fuel cell generates transdermal alcohol concentration signals.
